(19) United States
(12) Patent Application Publication
(10) Pub. No.: US 2009/0305322 A1
(43) Pub. Date: Dec. 10, 2009

(54) Isotope Detection and Uses Thereof

(76) Inventors: Eric L. Hegg, East Lansing, MI (US); Helen Kreuzer-Martin, Benton City, WA (US); David William Podlesak, Los Ausres, NM (US); Michael J. Lott, Bountiful, UT (US)

Correspondence Address: Ballard Spahr LLP, SUITE 1000, 999 PEACHTREE STREET, ATLANTA, GA, 30309-3915, US

(21) Appl. No.: 12/085,086

(22) PCT Filed: Nov. 15, 2006
(86) PCT No.: PCT/US06/44263

§ 371 Date: Aug. 3, 2009

Related U.S. Application Data
(60) Provisional application No. 60/737,614, filed on Nov. 16, 2005.
(60) Provisional application No. 60/851,191, filed on Oct. 12, 2006.

(51) Int. Cl. C12Q 1/02

(52) U.S. Cl. 435/29

(57) ABSTRACT

A method is disclosed for measuring the hydrogen and/or oxygen isotope ration of intracellular water in Escherichia coli cells and correlating the hydrogen and/or oxygen isotope ratio with the metabolic activity of the cell. A method is also disclosed for measuring the hydrogen and/or oxygen ratio of intracellular water via a probe, such as a fatty acid, and correlating the hydrogen and/or oxygen ratio with the metabolic activity of the cell. Methods for measuring the hydrogen and/or oxygen isotope ratio of water from eukaryotic organisms, such as cultured rat fibroblasts and whole mammals, and optionally relating the same to a metabolic rate, are also disclosed.

### I. CROSS-REFERENCE TO RELATED APPLICATIONS

This application claims the benefit of priority to U.S. Provisional Application No. 60/737,614, filed Nov. 16, 2005 and Application No. 60/851,191, filed Oct. 12, 2006, both of which are hereby incorporated herein by reference in their entirety.

### II. STATEMENT REGARDING FEDERALLY SPONSORED RESEARCH

This invention was made with government support under Grant GM66236 awarded by the National Institutes of Health and Grant 56200013 awarded by the Federal Bureau of Investigation. A portion of the work described herein was performed under the Laboratory Directed Research and Development Program at the Pacific Northwest National Laboratory, operated by Battelle for the U.S. Department of Energy under Contract DE-AC05-76RL01830. The government has certain rights in the invention.

### III. BACKGROUND

Cells can undergo numerous metabolic processes, many of which can alter intracellular water composition either directly, by generating new water molecules (e.g. dehydration reactions, respiration, etc.), or indirectly through the generation of CO₂ (whose oxygen atoms can rapidly exchange with water due to the activity of carbonic anhydrase) and other biomolecules such as carbohydrates capable of exchanging with water. These metabolic processes can result in intracellular water that is isotopically distinct from extracellular water.

Water can be transported into and out of cells through at least two different mechanisms. The principal mechanism by which water was believed to enter and exit a cell was via diffusion through the membrane. Although polar molecules, such as water, are generally unable to diffuse across biological membranes, the small size of a water molecule is believed to allow it to move through defects in the membrane as lipids diffuse laterally. Water can also be transported via aquaporins, or membrane channel proteins, at essentially diffusion-controlled rates. The rate at which these two processes can theoretically occur has led to the generally accepted assumption that intracellular water is isotopically indistinguishable from extracellular water. If the rate of one or more of these processes should vary, the isoopic composition of intracellular water could remain distinct from that of extracellular water. Any such variation could provide important information about the metabolic processes within the cell. While techniques exist to measure overall metabolic activity of an organism or an organ within an organism, no methods currently exist for measuring the metabolic activity of a single cell or small group of cells. Provided herein are compositions and methods for measuring the isotopic ratio of ²H/¹H and/or ¹⁸O/¹⁶O in intracellular water and thus assessing the metabolic activity of a cell.

### IV. SUMMARY OF THE INVENTION

In accordance with the purpose of this invention, as embodied and broadly described herein, this invention relates to compositions and methods for measuring the isotopic ratio of ²H/¹H and/or ¹⁸O/¹⁶O in intracellular water and assessing the metabolic activity of a cell. In this aspect, a method is provided for determining the metabolic rate of a cell by obtaining a cell that contains a quantity of intracellular water, and analyzing the intracellular water to determine the isotopic composition of the hydrogen and/or the oxygen from the intracellular water. This isotopic composition can subsequently be related to the metabolic activity of the cell.

In another aspect, a method is provided for measuring the isotopic ratio of ²H/¹H in a probe species and assessing the metabolic activity of a cell. In one aspect, the method of the present method comprises determining the isotopic ratio of hydrogen and/or oxygen in both intracellular and extracellular water, determining the percentage of isotopically distinct atoms, and determining the isotope ratio of metabolic water. In another aspect, the method comprises determining the isotope ratio of ²H/¹H in fatty acids, and relating such isotope values with intracellular water. In another aspect, the present method is suitable for use in assessing metabolic processes in bacterium, such as E. coli. In yet another aspect, the provided methods are suitable for assessing the metabolic processes of a subject, such as a rat, by, for example, examining rat fibroblast cells. In yet another aspect, the provided methods are suitable for assess the metabolic processes of a human subject. In a further aspect, the provided methods are suitable for examining the metabolic processes of a human subject via analysis of a fatty acid sample, such as that contained in a blood sample.

In another aspect, the provided methods can provide metabolic information useful for diagnosing metabolic anomalies, such as those observed in various cancers and weight disorders. In another aspect, the provided methods can provide useful information for elucidating the flow of protons through hydrogen evolving organisms. In another aspect, the provided methods can provide useful information on the metabolic rate of “mats” or other biofilms.

Additional advantages will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### V. BRIEF DESCRIPTION OF THE DRAWINGS

The accompanying drawings, which are incorporated in and constitute a part of this specification, illustrate several aspects described below. Like numbers represent the same elements throughout the figures.

FIG. 1 illustrates regression of the oxygen isotope ratio of extracted cell cake water versus that of growth medium water. Data is from five experiments in which cells were grown in 2×LB and harvested at mid-log phase. The standard errors of the slope and intercept are 0.019 and 0.21, respectively. Error bars representing the standard error of measurement for each data point are concealed within the symbols on the graph.

FIG. 2 illustrates regression of the calculated value of the oxygen isotope ratio of intracellular water as determined from the washing experiments versus the oxygen isotope ratio of the growth medium water. The slope of 0.29 indicates that 71% of the intracellular water was generated during metabolism, in agreement with the value calculated independently using the regression in FIG. 1.

FIG. 3 illustrates regression of the calculated hydrogen isotope ratio of rat cell fatty acid methyl esters versus that of culture water.

FIG. 4 illustrates regression of the calculated hydrogen isotope ratio of water derived from E. coli. cells versus that of culture water.

FIG. 5 illustrates the oxygen isotope ratio of water extracted from yeast cells grown in YPEG media of varying isotopic enrichment and harvested at either stationary or log phase. Slope of log phase graph=0.96508. Slope of stationary phase graph=0.97763. These data indicate that yeast in log phase have a smaller percentage of their water being identical to extracellular water. In other words, log phase cells have a greater percentage of intracellular water that is derived from metabolism.

FIG. 6 illustrates regression of the hydrogen isotope ratio of extracted cell cake water versus that of growth medium water. Data was pooled from five experiments in which the cells were grown at 37° C. in 2×LB and harvested during mid-log phase.

FIG. 7 illustrates regression of the calculated value of the hydrogen isotope ratio of intracellular water as determined from the washing experiments versus the hydrogen isotope ratio of the growth medium water. The slope of 0.33 (with a 95% confidence interval of 0.19) indicates that 48%-86% of the water was generated during metabolism, in agreement with the value calculated independently using the regression in FIG. 6.

FIG. 8 illustrates data from experiments with eight different lab rats. Four of the lab rats were raised on Salt Lake City (SLC) tap water and the remaining lab rats were raised on slightly enriched water. Five different tissue samples were collected from each of the rats. From the animals grown in slightly enriched water it can clearly be seen that the blood water has a very different isotope value for both O and H than the tissue water. The lab rats grown on SLC tap water follow the same trend but the values are much closer to each other (and are not separately labeled on this graph). This suggests that the isotope ratio of the metabolic water is reasonably close to that of SLC tap water. In the case of hydrogen, the signature of the food and tap water were similar thus masking the difference between metabolic water and extracellular water. Note, however, that tap water in other locations, such as, for example, Houston, can have different isotope ratios.

FIG. 9 illustrates rat fibroblasts harvested in either log or stationary phase. Cell cake water was extracted and both the H and O isotope ratio was determined. Significantly, slope is much bigger in the stationary (“stat”) phase cells than in the log (“exp”) phase cells. This demonstrates that some of the water comes from metabolism, and that as metabolism slows down, the percentage of metabolic water in the intracellular water also decreases.

FIG. 10 illustrates a repeat of the experiment shown in FIG. 9 in duplicate. The diamonds and the triangles are both log phase cell data. The squares and cross are both stationary phase cell data. The log phase cells have a slope around 0.81 while the stationary phase cells have a slope around 0.95. Again, this indicates that stationary phase cells have less metabolic water in their intracellular water than do log phase cells.

### VI. DETAILED DESCRIPTION

Before the present methods are disclosed and described, it is to be understood that the aspects described below are not limited to specific methods, as such may, of course, vary. It is also to be understood that the terminology used herein is for the purpose of describing particular aspects only and is not intended to be limiting.

Disclosed are materials, compounds, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an inhibitor is disclosed and discussed and a number of modifications that can be made to a number of R groups are discussed, each and every combination and permutation of the inhibitor and the modifications to its R group that are possible are specifically contemplated unless specifically indicated to the contrary. Thus, if a class of substituents A, B, and C are disclosed as well as a class of substituents D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-B, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

In this specification and in the claims which follow, reference will be made to a number of terms which shall be defined to have the following meanings:

It must be noted that, as used in the specification and the appended claims, the singular forms “a,” “an” and “the” include plural referents unless the context clearly dictates otherwise. Thus, for example, reference to a fatty acid includes two or more such fatty acids, mixtures of fatty acids, and the like.

“Optional” or “optionally” means that the subsequently described event or circumstance can or cannot occur, and that the description includes instances where the event or circumstance occurs and instances where it does not. For example, the phrase “optionally methylated” means that the substance can or can not be methylated and that the description includes both methylated and un-methylated embodiments.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

As used herein, a “wt. %”, “weight percent”, or “percent by weight” of a component, unless specifically stated to the contrary, refers to the ratio of the weight of the component to the total weight of the composition in which the component is included, expressed as a percentage.

As used herein, a “mole percent” or “mole %” of a component, unless specifically stated to the contrary, refers to the ratio of the number of moles of the component to the total number of moles of the composition in which the component is included, expressed as a percentage.

The term “ester” as used herein is represented by the formula —C(O)OA, where A can be an alkyl, halogenated alky, alkenyl, alkynyl, aryl, heteroaryl, cycloalkyl, cycloalkenyl, heterocycloalkyl, or heterocycloalkenyl group.

The term “cell”, as used herein, can refer to individual cells, cell lines, a primary culture, or cultures derived from such cells unless specifically indicated. A “culture” or “cell culture” refers to a composition comprising isolated cells of the same of a different type and can refer to a population of cells grown on or in a medium such as agar or LB broth.

A cell can be in vitro. Alternatively, a cell can be in vivo and can be found in a subject. A “cell” can be a cell from any organism including, but not limited to, a bacterium or a mammal.

As used herein, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. In one aspect, the subject is a mammal such as a primate or a human.

As used herein, “culture medium” or “growth medium” refer to a substance suitable for growing cells or cell cultures, such as, for example LB broth.

As used herein, a “probe” refers to a molecule or substance that is a product, at least in part, of a metabolic process, wherein a hydrogen, an oxygen, or a combination thereof is incorporated from water at a rate proportional to the rate of the metabolic process.

Stable isotope contents are expressed herein by “delta” notation as δ values in parts per thousand (%), where δ%=[(R_{A}/R_{Std})−1]*1000%, and R_{A} and R_{Std} are the molar ratios of the rare to abundant isotope (e.g. ²H/¹H) in the sample and the standard, respectively.

By the term “effective amount” of a compound as provided herein is meant a nontoxic but sufficient amount of the compound to provide the desired result. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The calculations and equations described herein are exemplary and are not intended to be limiting. Other mathematical formulae and approaches can be used to achieve the same result and the present invention is not intended to be limited to the specific equations and calculations recited.

The present invention provides methods for measuring the isotopic ratio of ² H/¹H and/or ^{18/16}O in intracellular water and thus, assessing the metabolic activity of a cell. The ability to assess such information can have a profound impact in the fields of biochemistry, cellular biology, and biogeochemistry.

In a general description, the present invention provides a method for determining the metabolic rate of a cell by obtaining a cell that contains a quantity of intracellular water, and analyzing the intracellular water to determine the isotopic composition of the hydrogen and/or the oxygen from the intracellular water. This isotopic composition can be correlated with metabolic activity of the cell using the steps described below.

An alternative method may be employed wherein a probe molecule, such as fatty acid, is analyzed in a similar manner and correlated with metabolic activity.

In an exemplary aspect, and not intended to be limiting, the general steps of the method comprise determining the isotopic ratio of hydrogen and/or oxygen in both intracellular and extracellular water, determining the percentage of isotopically distinct atoms, and determining the isotope ratio of metabolic water. In another aspect, the method comprises determining the isotope ratio of ²H/¹H in a probe, such as fatty acids, and correlating such isotope values with intracellular water.

In one aspect, the disclosed methods are suitable for use in assessing metabolic processes in bacterium, such as E. coli. In another aspect, the disclosed methods are suitable for assessing the metabolic processes of a subject, such as a rat, by, for example, examining rat fibroblast cells. In yet another aspect, the disclosed methods are suitable for assess the metabolic processes of a human subject. In a further aspect, the disclosed methods are suitable for examining the metabolic processes of a human subject via analysis of a sample comprising a probe, such as a fatty acid, such as that contained in a blood sample.

In another aspect, the disclosed methods can provide metabolic information useful for diagnosing metabolic anomalies, such as those observed in various cancers and weight disorders. In a specific aspect, the disclosed methods can provide metabolic information useful for diagnosing cancer such as lymphomas (Hodgkins and non-Hodgkins), B cell lymphoma, T cell lymphoma, leukemias, myeloid leukemia, carcinomas, carcinomas of solid tissues, squamous cell carcinomas, squamous cell carcinomas of the mouth, throat, larynx, and lung, adenocarcinomas, sarcomas, gliomas, high grade gliomas, blastomas, neuroblastomas, plasmacytomas, histiocytomas, melanomas, adenomas, hypoxic tumours, myclomas, AIDS-related lymphomas or sarcomas, metastatic cancers, mycosis fungoides, bladder cancer, brain cancer, nervous system cancer, lung cancers such as small cell lung cancer and non-small cell lung cancer, ovarian cancer, pancreatic cancer, prostate cancer, hepatic cancer, colon cancer, cervical cancer, cervical carcinoma, breast cancer, and epithelial cancer, renal cancer, genitourinary cancer, esophageal carcinoma, head and neck carcinoma, large bowel cancer, hematopoietic cancers, or testicular cancer.

Through the appropriate selection of cell and/or probe samples, a variety of disorders and/or metabolic variations can be identified, diagnosed, or monitored. One of skill in the art would be able to select an appropriate cell and/or probe sample to identify, diagnose, or monitor a specific metabolic disorder.

In another aspect, the disclosed methods can provide useful information for elucidating the flow of protons through hydrogen evolving organisms. In another aspect, the disclosed methods can provide useful information on the metabolic rate of “mats” or other biofilms.

It should be noted that not all of the steps and/or experiments described herein are required to assess metabolic activity. Many of the steps and experiments are not required and can be optionally performed to provide further data or refine existing data. The steps can be performed in any order that will provide the desired result.

A. Isotopically Distinct Intracellular Water

A large percentage of both the hydrogen and oxygen atoms in intracellular water can be derived from metabolic processes. According to the accepted mechanisms of heme O biosynthesis, the oxygen atom of the 17-hydroxyethylfarnesyl moiety is derived from water. Heme O molecules in Escherichia coli cells grown in 95% H₂¹⁸O, however, do not typically contain the expected quantity of labelled oxygen atoms, indicating that an additional source of water exists and that the isotope ratio of intracellular water can be different from extracellular water. The isotopic composition of, for example, ¹⁸O/¹⁶O in intracellular water can be determined with any suitable means capable of quantifying isotopically distinct atoms at the levels described herein, such as isotope-ratio mass spectrometry (IRMS). According to IRMS analysis, approximately 70% of the intracellular water oxygen atoms extracted from log-phase E. coli cells grown in 2×LB are derived from metabolic processes and can therefore be isotopically distinct from extracellular water.

While protons can diffuse across membranes independently from oxygen atoms (e.g. through proton channels), the hydrogen isotope ratio of intracellular water in E. coli cell can also be distinct from that of growth medium water, and thus, be a function of metabolic activity of the cell. Hydrogen from intracellular water can also be incorporated into certain molecules, such as fatty acids, during cell metabolism. These molecules can be used as probes to indirectly ascertain information on the metabolic processes within the cell. Any probe species that can incorporate an isotopically distinct atom during a metabolic process can be utilized in this method. One of skill in the art would be able to readily select an appropriate probe species.

In rapidly metabolizing organisms, intracellular water can be significantly different from extracellular water.

B. Isotope Ratio of Intracellular vs. Extracellular Water

Water molecules can enter a cell via diffusion from the culture medium water or be generated during metabolic reactions. An isotopic gradient of water can be maintained during the harvesting of cells by, for example, filtration, and the water extracted from the filtered cell cake can be modeled as a two-component mixture of medium and metabolic water in which

δ_{cell cake}=ƒ(δ_{medium})+(1−ƒ)(δ_{metabolic}), Equation 1

where δ_{cell cake}, δ_{medium}, and δ_{metabolic} are the hydrogen isotope ratios of the water extracted from the cell cake, the culture medium, and the metabolic water, respectively, and ƒ is the fraction of the cell cake water that is identical to the culture medium water. The composition of the culture medium, and thus, δ_{medium}, can be manipulated and δ_{cell cake} measured to yield a straight line of slope ƒ.

Cell cultures can be grown to a specific point or phase, for example mid-log phase, in a suitable medium, such as 2×LB, made with isotopically varying water. The mid-log phase cells can be harvested on filters and the resulting cakes removed and sealed for subsequent analysis and comparison to the spent culture medium. Water from both the cell cake and the spent medium can be analyzed as described herein to determine the isotope ratio of hydrogen, oxygen, or both, and determine the percentage of isotopically distinct atoms.

Cell cultures can also be grown and harvested after the cells have entered the stationary phase, such as approximately 12 hours post-inoculation, to determine the correlation of hydrogen isotope ratio with metabolic activity. The effect of metabolic rate on the isotope ratio can be further assessed by comparing intracellular water from cells grown at varying temperatures.

C. Percentage of Isotopically Distinct Atoms

The percentage of intracellular water isotopically distinct from growth medium water is presumed to derive from metabolism. As the cell cake can contain both intracellular and extracellular water, the extracted cell cake water can be modeled as a two-component mixture of intracellular and extracellular water:

δ_{cell cake}=ƒ(δ_{extracellular})+(1−ƒ)(δ_{intracellular}), (Equation 2)

where ƒ is the fraction of the cell cake water that is extracellular water, and δ_{extracellular} and δ_{intracellular} are the oxygen isotope ratios of the extracellular and intracellular water. If δ_{extracellular} is manipulated and δ_{cell cake} measured, Equation 2 becomes the equation of a straight line where the slope is equal to ƒ.

To further vary the composition of extracellular water, a cell culture can be grown to, for example, mid-log phase in a suitable medium, divided into multiple aliquots, and harvested on separate filters. The dry or semi-dry cell cakes can then be washed with fresh growth medium made with isotopically distinct water, replacing the extracellular water in the cake with the isotopically distinct wash water. The extracted water from the washed cell cakes can then be analyzed to determine δ²H and/or δ¹⁸O values and be regressed onto the wash water as depicted in Table 1 below:

This procedure can be performed by examining hydrogen, oxygen, or both. As the aliquots each contain the same percentage of intracellular water, no statistical difference should be expected between the samples for each of the hydrogen and oxygen analyses. This data can then be used to calculate the fraction of hydrogen and/or oxygen atoms in the intracellular water that derived from metabolic processes in log-phase cells. Similar experiments can be performed on cells harvested in stationary phase.

D. Determining the Isotope Ratio of Metabolic Water

At least two independent methods exist for calculating the isotope ratio (e.g. hydrogen) of intracellular water. The first method derives from Equation 1, [δ_{cell cake}=ƒ(δ_{medium})+(1−ƒ)(δ_{metabolic})], above. If δ_{medium} is manipulated, as described above, δ_{metabolic} can be calculated by dividing the intercept value by (1−ƒ).

The second method for estimating δ_{metabolic} uses data from the wash experiments, as described above. The δ²H value of intracellular water can be calculated and then, using Equation 2, the intercept value can be divided by (1−ƒ) to yield an estimate of the δ²H value of the intracellular water, as depicted in Table 1 below. Thus, the isotope ratio of the intracellular water can be represented as:

δ_{intracellular=(}h)δ_{growth medium}+(1−h)δ_{metabolic}, Equation 3

where h is the fraction of intracellular water that originated from the growth medium. A plot of the calculated δ_{intracellular} values versus the measured δ_{growth medium} values can yield a regression slope representing h, as illustrated in FIG. 7. The δ²H value of the metabolic water is equal to the y-intercept value divided by (1−).

E. Isotope Ratio of Atoms in Probes, such as Fatty Acids, Correlates with Intracellular Water

The percentage of intracellular water that is isotopically equivalent to culture medium water typically increases as the culture progresses from log to stationary phase. The difference in contribution from culture medium water to intracellular water can be reflected in the hydrogen isotope ratios of probes, such as fatty acids, that are biosynthesized during log phase or later in the life of the culture. For example, the probe can be a metabolic product that is specific to a tissue type or tumor. For example, the probe can be prostate specific antigen (PSA).

The isotopic relationship between culture water, nutrients, and lipids can be expressed in the equation

R_{fa}=ƒ_{water}α_{water}R_{water}+(1−ƒ_{water})αₙᵤₜᵣᵢₑₙₜₛRₙᵤₜᵣᵢₑₙₜₛ, Equation 4

where R_{fa}, R_{water}, and Rₙᵤₜᵣᵢₑₙₜₛ represent the hydrogen isotope ratios (R values) of the fatty acid, culture water, and nutrients, respectively; ƒ_{water} is the fraction of the fatty acid hydrogens that derive from water; and α_{water} and αₙᵤₜᵣᵢₑₙₜₛ (defined as R_{fa}/R_{water} and R_{fa}/Rₙᵤₜᵣᵢₑₙₜₛ) are the isotope fractionation factors between water and the fatty acid, and nutrients and the fatty acid, respectively. A regression of R_{fa} versus R_{water} can yield a line of slope ƒ_{water}α_{water} with an intercept of (1−ƒ_{water})αₙᵤₜᵣᵢₑₙₜₛRₙᵤₜᵣᵢₑₙₜₛ. If α_{water} is assumed to be relatively constant between log and stationary phases, then a change in the slope of the regression using the R values of fatty acids harvested from log- or stationary-phase cells can be ascribable to a change in ƒ_{water}.

Probes, such as fatty acids, can be prepared as described in the Examples below, from log-phase and/or stationary-phase cells, such as those above. Depending upon the analysis method, the specific preparation method of a fatty acid can vary. In one aspect, the fatty acid sample can be methylated. In this aspect, the methylated fatty acids can be analyzed via Gas Chromatography-Mass Spectrometry (GC-MS) to identify the fatty acid methyl ester components. The hydrogen isotope ratios of individual fatty acids can then be determined by GC-IRMS. Other preparation and/or derivation steps can be used to render the probe species in a suitable form for analysis. One of skill in the art would be able to select an appropriate preparation method for a probe species. The data illustrated in the Examples below demonstrates that a significant fraction of the intracellular water in log-phase cells grown in 2×LB can derive from metabolic processes. The isotope ratio of metabolic water is also reflected in fatty acids, indicating that metabolites can be used as an indirect probe for metabolic activity in living cells.

F. Energy Balance and Metabolism

Doubly-labeled water is commonly used by researchers to study metabolism and energy balance of humans and animals. After an initial dose of ²H₂¹⁸O, ²H is eliminated only as water and ¹⁸O is eliminated as water and CO₂. The difference in elimination rates between the ²H and the ¹⁸O is a measure of CO₂ production. CO₂ is the final product of energy catabolism within the cell. Thus, estimates of CO₂ production can be used to investigate energy balance and metabolism. At this time, the influence of diet composition on energy balance is not clear. Funding agencies such as NIH are interested in further investigating energy balance and metabolism in humans because of the increased rates of obesity in the US. To this end, researchers continue to use doubly-labeled water to investigate energy balance in humans and in model animals such as laboratory rats. The herein disclosed ability to measure the isotopic composition of the body water pool of the whole animal and the isotopic composition of the tissue water, where metabolism directly occurs, allows for an increase in the understanding of how diet composition directly influences energy balance. Likewise, the disclosed ability to measure differences in the isotopic composition of the tissue water can allow a better understanding of the mechanisms involved in excessive weight gain

### G. EXAMPLES

The following examples are put forth so as to provide those of ordinary skill in the art with a complete disclosure and description of how the compounds, compositions, articles, devices, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Oxygen Isotopes Indicate Most Intracellular Water in Log-Phase Escherichia Coli is Derived from Metabolism

### Materials and Methods

Cultures: E. coli BL21 (DE3) cultures were grown in 2×Miller Luria-Bertani (LB) broth (EMD Chemicals) at 37° C. with shaking at 225 rpm. The culture volume was one-tenth the flask volume. The 2××LB contained 20 g tryptone, 10 g yeast extract, and 20 g NaCl per liter and had an osmolality of 838 mosmol/kg, as measured on a Wescor 5500 vapor pressure osmometer. Batches of LB were made with isotopically varying water.

To determine the optical density readings associated with mid-log phase, growth of cultures were monitored in 2×LB, regular absorbance measurements taken at 600 nm on a Hach Odyssey spectrophotometer. Mid-log phase was associated with OD₆₀₀ readings of 0.8 to 1.0 and a doubling time of approximately 30 minutes. Stationary phase cells were harvested about 12 hours after inoculation, when the OD₆₀₀ of a 1:10 dilution of the culture had been about 0.7 for 3 hours. Cell cakes were harvested by pouring the culture through a 0.2 μm NL 16 filter (Schleicher and Schuell, Dassel, Germany) under house vacuum. As soon as the cell cake appeared dry, it was either harvested by scraping it from the membrane with a razor blade or washed with 1 mL of 2×LB made with isotopically varying water and then harvested. The cell mass was transferred immediately to a vial, sealed, and frozen. Samples of spent medium and wash solutions were collected and frozen at the same time.

Water was extracted cryogenically from the cell pellets and spent medium samples using a modification of earlier methods (Ingraham, N. L. & Shadel, C. 1992; Araguas-Araguas, L., et al. 1995). Vials containing frozen cell pellets or medium were placed inside a heavy-walled Pyrex test tube connected to a cold finger trap. The Pyrex tube and cold finger assembly was connected to a vacuum line, but kept isolated from the vacuum pump via a valve. The test tube containing the sample vial was then submerged in liquid nitrogen for several minutes, following which the valve to the vacuum pump was opened and the assembly evacuated. The test tube and cold finger were then re-isolated from the vacuum pump and checked for constant vacuum with an in-line vacuum gauge. Water was then completely distilled from the sample and collected in the cold finger under static vacuum by placing the Pyrex tube containing the sample vial into a boiling water bath for about an hour. Once distillation was complete, the distilled water was placed in crimp-top vials, sealed, and stored in a cold room prior to stable isotope ratio analysis.

Stable isotope ratio measurements. Stable isotope ratios were measured relative to internationally recognized standards. Laboratory standards were calibrate to the international standards, and then the laboratory standards included as internal standards in every run. Stable isotope contents are expressed in “delta” notation as δ values in parts per thousand (%), where δ%=(R_{A}/R_{Std}−1)*1000% and R_{A} and R_{Std} are the molar ratios of the rare to abundant isotope (e.g. ¹⁸O/¹⁶O) in the sample and the standard. The standard used for both oxygen and hydrogen is Vienna Standard Mean Ocean Water [VSMOW] (Coplen, T. B. 1996).

Oxygen stable isotope ratios were determined on a ThermoFinnigan-MAT Delta Plus XL isotope ratio mass spectrometer (ERMS, Bremen, Germany) equipped with a Thermo Chemical Elemental Analyzer (ThermoFinnigan-MAT, Bremen Germany) and a GC-PAL autosampler (CTC Analytics, AG, Zwingen, Switzerland) (Gehre, M., et al. 2004). Injection volume was 0.5 μl. Water samples were analyzed in duplicate and the results averaged. The average standard deviation of repeated measurements of water standards was 0.2%.

Results

Cellular Water Can Be Isotopically Distinct from Growth Media Water: Water molecules can enter a cell via diffusion from the culture medium water or be generated during metabolic reactions. If an isotopic gradient could be maintained during harvesting of a cake of cells on a filter, then water extracted from the cell cake could be modeled as a two-component mixture in which

δ_{cell cake}=ƒ(δ_{medium})+(1−ƒ)(δ_{metabolic}), (Equation 1)

where δ_{cell cake}, δ_{medium}, and δ_{metabolic} are the oxygen isotope ratios of the water extracted from the cell cake and the culture medium, and of the metabolic water, respectively, and ƒ is the fraction of the cell cake water that is identical to the culture medium water. If δ_{medium} is manipulated and δ_{cell cake} measured, Equation 1 becomes the equation of a straight line where the slope is equal to ƒ.

Four cultures of E. coli were grown to mid-log phase in 2×LB medium made with isotopically varying water, and the cells harvested on filters. The cell cakes were then scraped from the filters, sealed in vials, and frozen. Samples of the spent medium were also collected. Water was extracted from both the cell cakes and the spent medium and the oxygen isotope ratios determined. This experiment was conducted five times.

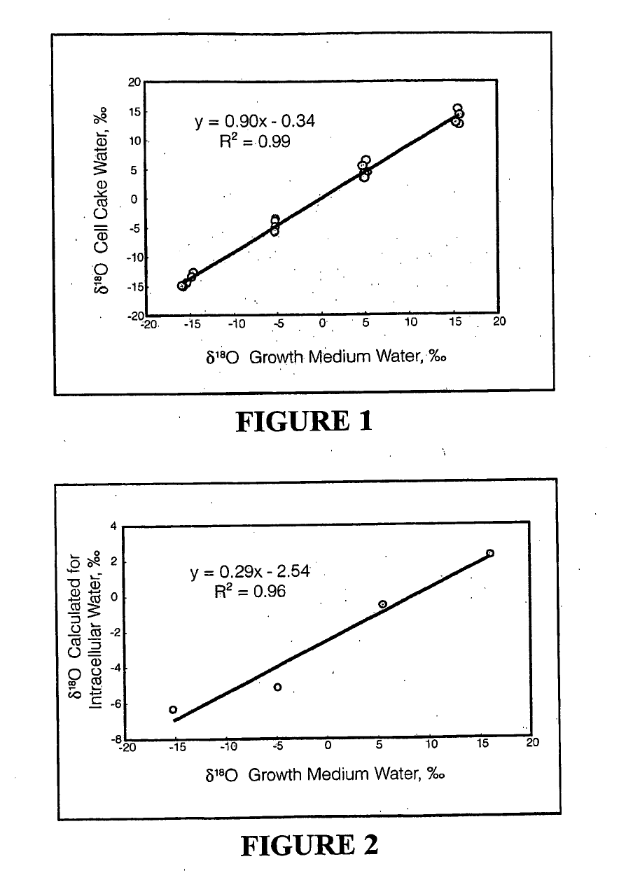

The slopes of δ_{cell cake} versus δ_{medium} values obtained in the five experiments were not significantly different (F=1.03, where F_{0.05}=3.63) (Sokal, R. R. & Rohlf, F. J. 1995), and the data from the five experiments were therefore combined (FIG. 1). The slope of the regression line of the pooled data is 0.90 (Table 1). Thus, the water extracted from the cell cakes was isotopically distinct from the growth medium water. One way in which the cell cake water could become different from the growth medium water would be if significant evaporation occurred as the cells were being collected on the filters, since evaporation generally increases the isotope ratios of the residual water (Farquhar, G. D. & Lloyd, J. 1993; Farquhar, G. D., et al. 1993; Farquhar, G. D. & Cernusak, L. A. 1989). These data are not consistent with evaporative enrichment, however, because the cell cake water samples extracted from cells grown in media made with isotopically heavy water were depleted when compared to the medium water. This difference is unlikely to be accounted for by evaporation and is instead more consistent with the two-component mixing model of Equation 1. The slope of 0.90 indicates that approximately 10% of the oxygen atoms extracted in the cell cake water were isotopically distinct from the growth medium water.

The Presence of Isotopically Distinct Water Is Correlated with Metabolic Activity: To test whether this isotopically distinct water was generated from metabolism, two experiments were performed in which water was extracted from cells that were less metabolically active than cells harvested in log phase. It was expected that water from less metabolically active cells would have a lower percentage of oxygen generated from metabolism, and that the slopes of the cell cake versus medium graphs would therefore be higher than 0.90. In the first test, cells were harvested at stationary phase (after ˜12 hours of growth). The slopes of the regression lines from the two experiments were each 0.96 (Table 1). A statistical comparison of the pooled stationary-phase experiments (Table 1) to the pooled data from the log-phase experiments showed that the slopes were significantly different.

TABLE 1
Regression statistics of extracted cell cake water versus growth medium water for cells
harvested under different conditions

| | | | | | P value at which |
|---|---|---|---|---|---|
| | | | | Number of | the slope is |
| Temperature and | | | | experiments | different from the |
| growth stage at | Slope of | R² value of | Standard | (four cultures | 37° C. log-phase |
| time of harvest | regression | regression | error of slope | per experiment) | experiments* |
| 37° C., log phase | 0.90 | 0.99 | 0.019 | 5 | — |
| 37° C., stationary | 0.96 | 0.99 | 0.011 | 2 | 0.01^{†} |
| phase | | | | | |
| 6° C., log phase | 0.98 | 0.99 | 0.015 | 1 | 0.05^{‡} |

*Confidence level determined in an F test.
^{†}F = 8.38, F_{0.01} = 7.88
^{‡}F = 7.60, F_{0.05} = 4.38

In the second test, cultures were grown to mid-log phase at 37° C. as before, but then each culture split into two equal parts. One part was harvested immediately, while the second part was transferred to a chilled flask and put on a shaker at 6° C. for 90 minutes prior to harvesting. The optical density of the chilled cultures only increased from 1.08 to 1.12 during this time. The slope of the regression line of water extracted from the 37° C. cells versus medium water was 0.90, confirming that the relationship between intracellular water and growth water in this experiment was the same as in the five log-phase experiments described above. The slope of the regression line of the 6° C. cell cake water versus medium water, however, was 0.98, significantly different from the 37° C. log-phase data (Table 1). Thus, when cells are chilled prior to harvesting, only 2% of the oxygen atoms of the total cell cake water are isotopically distinct from the medium.

The results from both of these tests indicate that some of the water extracted from the cell cakes was a product of cellular metabolism. In both cases, slowing the metabolic rate of the culture, either by allowing the cultures to go to stationary phase or by chilling the cells, resulted in a smaller contribution of metabolic water to the total cell cake water. This reduction was reflected in the larger slopes of the cell cake water versus medium water relationships.

Approximately 70% of Intracellular Water in Log-Phase E. coli Cells Is a Product of Metabolism. The data indicate that 10% of the total water extracted from log-phase cells was generated by metabolism, but that total pool of water was itself a mixture of extracellular and intracellular water. To determine what percentage of the intracellular water was metabolic water, it first had to be determine what percentage of the total cell cake water was intracellular.

The relationship between the isotope ratios of the total cell cake water, extracellular water, and intracellular water can be expressed as follows:

δ_{cell cake}=ƒ(δ_{extracellular})+(1−ƒ) (δ_{intracellular}), (Equation 2)

where ƒ is the fraction of the cell cake water that is extracellular water, and δ_{extracellular} and δ_{intracellular} are the oxygen isotope ratios of the extracellular and intracellular water. If δ_{extracellular} is manipulated and δ_{cell cake} measured, Equation 2 becomes the equation of a straight line where the slope is equal to ƒ.

A culture of E. coil was therefore grown to mid-log phase in 2×LB, the culture split into four aliquots and immediately harvested on separate filters. The cell cakes were then washed with fresh 2×LB made with isotopically distinct water. This washing procedure replaced the extracellular water in the cake, and the isotope ratios of the water in the 2×LB used to wash the cell cakes (δ_{wash solution}) was therefore equal to δ_{extracellular} in Equation 2. Water was extracted from the washed cell cakes and the wash solutions, their δ¹⁸O values measured, and the cell cake water values regressed onto the wash water (Table 2). This experiment was conducted four times, varying the isotopic composition of the growth medium water. An F-test showed that the slopes of the regression lines were not significantly different. The average slope was 0.86 (Table 2), indicating that 14% of the total cell cake water was intracellular.

TABLE 2
Regression statistics of extracted cell cake water wash water

| | Slope of extracted | | | Calculated δ¹⁸O |
|---|---|---|---|---|
| δ¹⁸O of growth | cell water versus | R² of | Y intercept | of intracellular |
| medium water, ‰ | wash water | regression | value^{†} | water, ‰*^{†} |
| −15.2 | 0.81 | 0.99 | −1.2 | −6.32 |
| −4.9 | 0.85 | 0.99 | −0.77 | −5.13 |
| 5.5 | 0.93 | 0.99 | −0.44 | −0.55 |
| 16.1 | 0.86 | 0.99 | +0.32 | 2.26 |
| | Average = 0.86^{‡} | | | |
| | Standard Error = 0.025 | | | |

*The δ¹⁸O value of intracellular water = (y intercept)/(1 − slope). Intracellular water is itself a combination of growth medium water and intracellular water as described by Eq. 3.
^{†}Average and standard error are not presented, because the y intercept values and the δ¹⁸O values of the intracellular water are expected to be different as a result of the different δ¹⁸O values of the growth medium waters.
^{‡}These slopes are not statistically different (F = 1.59; F_{0.05} = 4.35).

The fraction of intracellular water derived from metabolism is equal to the fraction of total cell cake water derived from metabolism (0.10, FIG. 1) divided by the fraction of total cell cake water that is intracellular (0.14, Table 2). Therefore, approximately 71% of the oxygen atoms in intracellular water extracted from log-phase cell cakes originated from metabolism. The total error in this estimation is 19% when the standard error of the two slopes (0.019 for metabolic water and 0.025 for intracellular water) are used in a propagation of errors calculation (Shoemaker, D. P., et al. 1989). A 71% estimate of the oxygen atoms in intracellular water being derived from metabolism is consistent with and explains previous in vivo heme O labelling results (Brown, K. R., et al. 2003).

Two wash experiments were also performed in which the cells were allowed to go to stationary phase before harvesting. A comparison of these data to that from the log-phase harvests showed no significant difference in slopes ( =0.08, F_{0.05}=4.84). Therefore, the result reported above in which the water extracted from stationary-phase cell cakes had a smaller contribution from metabolic water was not a consequence of a greater contribution of extracellular water to the cell cake water.

The δ¹⁸O value of metabolic water can be estimated by two independent methods using either the data from the growth experiments or the data from the washing experiments. According to Equation 1, the y-intercept term is equal to (1−f)δ_{metabolic}, where ƒ is the slope of the line and δ_{metabolic} is the oxygen isotope ratio of the metabolic water. In FIG. 1, the y-intercept value from the growth experiments is −0.34 and the slope is 0.90, yielding a predicted oxygen isotope ratio for the metabolic water of −3.4%.

Estimating the δ¹⁸O value of metabolic water from the washing experiment data requires that the δ¹⁸O value of intracellular water first be calculated. In Equation 2, the y-intercept from the washing experiments is equal to (1−ƒ)(δ_{intracellular}) where ƒ is the slope of the line. Thus, dividing the intercept value by (1−ƒ) yields an estimate of the δ¹⁸O value of the intracellular water (Table 2). According to the disclosed model, the isotope ratio of the intracellular water can be represented as:

δ_{intracellular}=(g)δ_{growth medium}+(1−g)δ_{metabolic}, (Equation 3)

where g is the fraction of intracellular water that originated from the growth medium. A plot of the calculated δ_{intracellular} values versus the measured δ_{growth medium} values yielded a regression slope of 0.29, representing g (FIG. 2). The δ¹⁸ O value of the metabolic water is equal to the y-intercept value divided by (1−g). This value is −3.6%, almost identical to the value estimated from the data in FIG. 1 but derived using independent data.

Significantly, the data from the washing experiments also support the previous estimate of the fraction of intracellular water derived from metabolism. From Equation 3, the fraction of intracellular water generated from metabolism is equal to (1−g), or 0.71. This estimation that 71% of intracellular water is derived from metabolism is identical to that reached using the slope of the regression in FIG. 1.

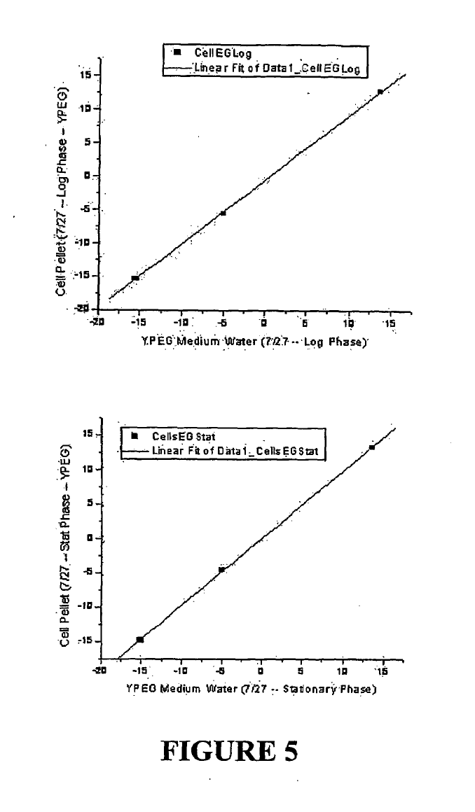

Similar tests and results have been performed with eukaryotic cells and are represented in FIG. 5.

### Example 2

### Metabolic Processes Account for the Majority of the Intracellular Water in Log-Phase Escherichia coil Cells as Revealed by Hydrogen Isotopes

Cell Cultures: E. coli BL21 (DE3) cultures were grown in 2×Miller Luria-Bertani (LB) broth (FMD Chemicals) at 37° C. to either mid-log or stationary phase. The cells were then collected via filtration, transferred to a vial, sealed and frozen. Water was then extracted cryogenically from the cell pellets and spent medium samples. The desiccated cell pellets were stored at room temperature prior to lipid extraction.

Fatty Acid Extraction and Analysis: Fatty acids were extracted from desiccated cell pellets, such as those prepared in Example 1, by saponification and then converted to methyl esters for structural analysis by gas chromatography/quadrupole mass spectrometry (GC-MS) and for isotope ratio measurements by gas chromatography-isotope ratio monitoring mass spectrometry (GC-IRMS). The extraction and methylation were performed in glassware that had been baked at approximately 500° C. for approximately 8 h to remove organic contamination. All aqueous solutions were extracted 5 times with hexane prior to use, and organic solvents were of the highest grade and used without further purification. Control blank extractions showed no contamination when analyzed by GC-MS.

Preparation of Fatty Acid Methyl Esters: Desiccated cell pellets were saponified in 5 mL of 0.5 M NaOH for 2 h at 70° C. in 16×125 mm test tubes with Teflon-lined caps. The solution was then acidified to a pH of 3-6 by the dropwise addition of 4 M HCl. 2.5 mL of an aqueous 5% NaCl solution was added, and the mixture was extracted 3 times with methyl tert-butyl ether (MTBE). The extracted organic layers were combined in a pear-shaped flask and the majority of the MTBE removed by rotary evaporation. The remaining solution was transferred to a borosilicate glass vial and evaporated to dryness under a stream of N₂. 1 mL of approximately 3% BF₃ in anhydrous methanol (Burdick and Jackson, Muskegan, Mich.) was added to the vial, which was capped with a Teflon-lined cap and sealed with Teflon tape. Methylation reactions were incubated for 2 h at 100° C. The reaction mixture was transferred to a 16×125 mm test tube. The vial was rinsed 3 times with methanol and 3 times with hexane, with the rinse solutions added to the test tube. 2 mL of an aqueous 5% NaCl solution was added to the tube and the mixture was extracted three times with 3 mL hexane. The volume of the combined organic layers was subsequently reduced to approximately 100-200 μL by evaporation under a stream of N₂. The identity of major components of the mixtures was determined by GC-MS analysis of 1 μL samples on a ThermoFinnigan Trace GC-MS equipped with a 30 m DB5 column.

Stable-Isotope Ratio Measurements: Stable-isotope ratio measurements were made at the Stable-Isotope Ratio Facility for Environmental Research at the University of Utah in Salt Lake City. Stable-isotope ratios were measured relative to Vienna Standard Mean Ocean Water (VSMOW), an internationally recognized standard. Laboratory standards were calibrated to the VSMOW standard, and included as internal standards in every analysis. Stable isotope contents are expressed in “delta” notation as δ values in parts per thousand (%), where δ%=[(R_{A}/R_{Std})−1]*1000%, and R_{A} and R_{Std} are the molar ratios of the rare to abundant isotope (e.g. ²H/¹H) in the sample and the standard, respectively. The δ notation is non-linear with respect to isotopic abundances, which can lead to large errors in calculation based on δ values if the range in isotope ratios is large, as is often the case with H. All calculations were made using R values and were reported in δ values. No difference was observed in the slopes calculated based on R or δ values at the level they reported.

Analysis of Water Samples: The hydrogen stable isotope ratios of water samples were determined on a ThermoFinnigan-MAT Delta Plus XL isotope ratio mass spectrometer (IRMS, Bremen, Germany) equipped with a Thermal Conversion Elemental Analyzer (TCEA, ThermoFinnigan-MAT, Bremen Germany) and a GC-PAL autosampler (CTC Analytics, AG, Zwingen, Switzerland). The injection volume was 0.5 μL. Water samples were analyzed in duplicate and the results averaged. The average standard deviation of repeated measurements of water standards was 2%.

Analysis of Lipid Samples: Stable hydrogen isotope ratios of lipids were measured on a ThermoFinnigan-MAT Delta Plus XL IRMS equipped with a Hewlett-Packard GC with a 30 m DB1 column coupled to a GC-TCIII interface. In this instrumental configuration, samples were injected into the GC and components of the mixture separated on the GC column. The separated components enter the pyrolysis reactor sequentially and their hydrogen atoms are converted to H₂ gas. Each peak of H₂ enters the IRMS where its isotope ratio is determined; thus the hydrogen isotope ratio of each well-separated compound present in sufficient quantity can be measured.

An instrumental correction for H₃ was determined daily from injections of a standard alkane mixture. The standard alkanes and fatty acid isotope ratio values were standardized against pulses of reference hydrogen gas (δ²H=−202.45%) injected at the beginning and end of every run. The average absolute error of measurements of the isotope ratio values of the individual standard alkane peaks was 4.5%, with a standard deviation of 4.2.

The correction factor for the three hydrogen atoms added to the fatty acids during the methylation step was determined by measuring the hydrogen isotope ratio of a 9:0 fatty acid purchased from Alltech (Deerfield, Ill.) by direct injection into the TCEA, as described above for water. The fatty acid was then methylated using the procedure described above and the hydrogen isotope ratio of the fatty acid methyl ester measured by GC-pyrolysis-mass spec. By comparing the hydrogen isotope ratios of the methylated and un-methylated forms of the fatty acid, it was determined that the % ²H value of the three hydrogen atoms added during methylation was −100%. This calculation ignored the hydrogen atom on the carboxylic acid group, the isotope ratio of which could not be separately measured because it would have been lost during the methylation procedure. The δ²H value of the fatty acid is assumed to be a function of its 17 alkyl hydrogen atoms that contributed to the value of the ester. The ignored hydrogen atom was one of 18, is not expected to contribute significantly to the experimental error, and would not alter the correlation between the isotope ratios of the fatty acids and growth medium.

Cell cultures grown to mid-log and stationary phase: Four cultures of E. coli were grown to mid-log phase in 2×LB medium made with isotopically varying water, and the cells harvested on filters. The cell cakes were then scraped from the filters, sealed in vials, and frozen. Samples of the spent medium were also collected. Water was extracted from both the cell cakes and the spent medium and the hydrogen isotope ratios determined. This experiment was conducted five times.

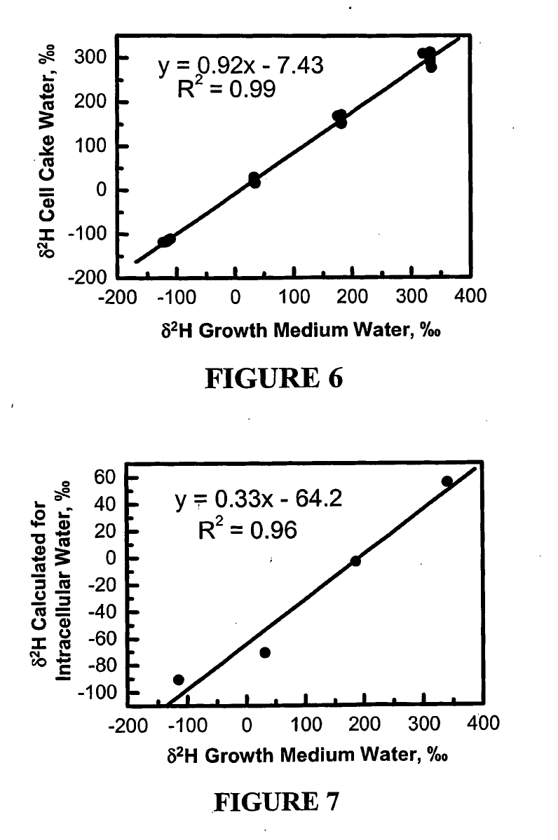

The slopes of δ_{cell cake} versus δ_{medium} values obtained in the five experiments were not significantly different (F=0.19, where F_{0.05}=3.48), and the data from the five experiments were combined, as illustrated in FIG. 6. The slope of the regression line of the pooled data is 0.92 with a 95% confidence interval of 0.03. This result suggests that approximately 8% of the hydrogen atoms in extracted cell cake water were isotopically distinct from extracellular water. This result is consistent with oxygen analysis that indicates that about 10% of the oxygen atoms in similar samples were metabolic. The average slope of the oxygen experiments was 0.90 with a 95% confidence interval of 0.04. The average slopes of the oxygen and hydrogen regressions are not significantly different (F=0.06 where F_{0.05}=4.1).

This experiment was repeated to determine the correlation between the hydrogen isotope ratio of intracellular water and metabolic activity. In the repeated experiment, the cells were harvested after they had entered stationary phase, at about 12 hours post-inoculation. The slope of the pooled data from two trials was 0.965, significantly different from the log-phase slope (F=16.8 where F_{0.01}=7.8). The results of this experiment indicate that when E. coli cells were harvested in stationary phase, 3.5% of the hydrogen atoms in the extracted cell cake water were isotopically distinct from growth medium water instead of 8%, very similar to oxygen isotope ratio experiments where the average slope of the stationary phase experiments was 0.961.

In an additional experiment, the effect of metabolic rate was assessed by comparing intracellular water from cells grown at different temperatures. Two identical cultures were prepared; one was incubated at the standard 37° C. temperature while the second was incubated at 18° C. The cells were harvested at log phase, and the water was cryogenically extracted in a fashion identical to that above; the only difference between the two cultures being the incubation temperature and therefore the metabolic rate. A plot of cell cake water versus growth medium water yielded a slope that was significantly larger for the 18° C. cells than for the 37° C. cells, indicating that a substantially smaller fraction of intracellular water is isotopically distinct from growth medium water when the cells are incubated at a reduced temperature. Together, these data are consistent with the hypothesis that the isotopically distinct hydrogen atoms are derived from metabolism.

Percentage of Isotopically Distinct Hydrogen Atoms: To determine the percentage of intracellular water that was isotopically distinct from growth medium water and presumably derived from metabolism, it is necessary to account for the fact that water extracted from a cell cake can contain both intracellular and extracellular water.

A culture of E. coli was grown to mid-log phase in 2×LB, at which time the culture was split into four aliquots and immediately harvested on separate filters. As soon as the cell cakes appeared dry on the filters, they were washed with fresh 2×LB made with isotopically distinct water. This washing procedure replaced most of the extracellular water in the cake, and the isotope ratios of the water in the 2×LB used to wash the cell cakes (δ_{wash solution}) was therefore equal to δ_{extracellular} (as in Equation 2 above). It is preferred to replace all of the extracellular water in the cake to prevent error. Water was extracted from the washed cell cakes and the wash solutions, and the δ²H values subsequently measured. The cell cake water values were then regressed onto the wash water as illustrated in Table 3 below. This experiment was conducted four times, varying the isotopic composition of the growth medium water. An F-test showed that the slopes of the regression lines were not significantly different.

TABLE 3
Regression statistics from washing experiments:
Extracted cell cake water versus wash water.

| | | Slope of δ²H extracted | | y | Calculated δ²H of |
|---|---|---|---|---|---|
| Growth | δ²H culture | cell cake water versus | R² of | intercept | intracellular water, |
| phase of cells | water, ‰ | wash water | regression | value | ‰^{†,‡} |
| Log | −115 | 0.80 | 0.99 | −18.1 | −90.5 |
| Log | 32 | 0.85 | 0.99 | −10.6 | −70.7 |
| Log | 187 | 0.90 | 1.0 | −0.3 | −3.0 |
| Log | 342 | 0.86 | 0.99 | 7.8 | 55.7 |
| Average* | | 0.85; SE = 0.21 | | | |
| Stationary^{§} | −120 | 0.88 | 0.99 | −17.4 | ND |
| Stationary^{§} | −119 | 0.87 | 1.0 | −14.3 | ND |

SE: standard error
ND: not determined
*Average slope of log-phase experiments; SE = standard error. The slopes of the individual log-phase experiments are not statistically different [F = 0.88 where F_{0.05} = 4.35].
^{§}The slopes of the stationary-phase experiments were not statistically different from the log-phase experiments (F = 0.56 where F_{0.05} = 4.1).
^{†}The δ ²H/¹H value of intracellular water = (y intercept)/(1 − slope) in Equation 2. Intracellular water is itself a combination of growth medium water and metabolic water as described by Equation 3.
^{‡}Average and standard error are not presented because the y-intercept values and the δ ²H/¹H values of the intracellular water are expected to be different due to the different δ ²H/¹H values of the growth medium waters.

The average slope was 0.85 (Table 1), indicating that 15% of the total cell cake water was intracellular. This result was similar to oxygen experiments, where the average slope of washing experiments was 0.86. An F test showed that the slopes of the oxygen and hydrogen regressions of wash water onto extracted cell cake water were not statistically different (F=0.005 where F_{0.05}=4.1). Two experiments with stationary-phase cells yielded slopes that were statistically indistinguishable from the slopes obtained with log-phase cells, indicating that the same percentage of cell cake water was intracellular when the cells were harvested at stationary phase (F=0.56, where F_{0.05}=4.1).

The fraction of hydrogen atoms in intracellular water that derived from metabolism in log-phase cells can then be calculated as 0.08 (the fraction of hydrogen atoms that were distinct from medium water; FIG. 6)/0.15 (the fraction of hydrogen atoms that were intracellular; Table 1)=0.53, or 53%. The total error in this estimation is 12% when the standard error of the two slopes (0.014 for metabolic water and 0.021 for intracellular water) is used in a propagation of errors calculation. Oxygen isotope analysis showed that approximately 71% of the oxygen atoms in intracellular water derived from metabolism during log-phase growth, with a total error in the estimate of 19%.

At stationary phase, the slope of the extracted cell cake water versus medium water was 0.965. Thus, the fraction of cell cake water derived from metabolism is 0.035/0.15, or 23%. The total error in this estimation is 5% when the standard error of the two slopes (0.007 for the stationary phase metabolic water and 0.014 for the intracellular water) are used in a propagation of errors calculation. Again, this compares well with oxygen analysis data that indicated only ˜29% of the oxygen atoms in intracellular water were derived from metabolism in stationary-phase E. coli cells.

Calculating the Hydrogen Isotope Ratio of Metabolic Water: In the growth experiments described above, δ_{medium} was manipulated so that the slope of the graph in FIG. 6 would be equal to ƒ and the intercept equal to (1−ƒ)(δ_{metabolic}). Therefore, δ_{metabolic} can be calculated by dividing the intercept value, −7.43, by (1−71 ), giving a result of about −93%. The total error in this estimate is about 40% as determined in a propagation of errors calculation using the standard errors of measurement of the slope (0.014) and the intercept (2.8).

The second method for estimating δ_{metabolic} uses data from the wash experiments. A plot of the calculated δ_{intracellular} values versus the measured δ_{growth medium} values yielded a regression slope of 0.33, representing h (FIG. 7). The δ²H value of the metabolic water is equal to the y-intercept value divided by (1−h). This value is −96%, almost identical to the value estimated from the data in FIG. 6, but derived using independent data.

The data in FIG. 7 is also consistent with the estimate of the fraction of intracellular water that is derived from metabolism. From Equation 3, that fraction of intracellular water is equal to (1−h), or 0.67. The 95% confidence interval for the slope shown in FIG. 7 is 0.19, giving a range of values for (1−h) of 0.48-0.86, consistent with the previous estimate of 0.53±0.11. When data from the stationary-phase growth experiments was used (slope=0.965; y-intercept=6.38), a value of −179% for δ²H of metabolic water at stationary phase was calculated. The total error in this calculation is 54.5% (standard error of slope=0.007 and of intercept=1.4) as determined by a propagation of errors calculation.

Correlation of Isotope Ratio in Fatty Acids with Intracellular Water: According to the model described herein, if approximately 53% of the hydrogen atoms in log-phase intraceflular water originate from metabolic activity, then the remaining 47% are equivalent to the culture medium water. Likewise, in stationary phase about 23% of the hydrogen atoms originate from metabolic activity, and the remaining 77% are equivalent to culture medium water. Presumably, the percentage of intracellular water that is isotopically equivalent to culture medium water increases as the culture progresses from log to stationary phase, and thus, the difference in contribution from culture medium water to intracellular water would be reflected in the hydrogen isotope ratios of fatty acids biosynthesized during log phase or later in the life of the culture.

Fatty acids were prepared and methylated from the cell pellets of the experiments above. These samples comprised two independent sets of four log-phase and four stationary-phase cultures produced in 2×LB made with isotopically varying water (16 total cultures). The hydrogen isotope ratio of the growth medium water at the time the cells were harvested had previously been determined.

The identity of various fatty acid methyl ester peaks was established by GC-MS. The hydrogen isotope ratios of individual fatty acids was determined by GC-IRMS, making a minimum of three independent measurements of each preparation. The average standard deviations of the triplicate measures of 14:0 and 16:0 fatty acid methyl esters from all four preparations was 3.4%. A comparison of the slopes of the regressions of R_{fa} vs R_{water} of 14:0 and 16:0 fatty acids isolated from log- and stationary-phase cells (Table 4) shows that the slope of the regression of R_{fa} onto R_{water} is significantly greater in stationary phase. In other words, a greater percentage of the hydrogen atoms in 14:0 and 16:0 fatty acids are derived from extracellular water when the cells are in stationary phase, or conversely, fatty acids in log-phase cells contain more water from metabolic water.

TABLE 4
Regression data of R_{fatty acid} versus R_{medium water} of 14:0
and 16:0 fatty acids prepared from two independent sets each of
log-phase and stationary-phase cells.

| | Set A | | Set B | | A and B | |
|---|---|---|---|---|---|---|
| Fatty acid/growth phase | Slope | SE^{⊥} | Slope | SE^{⊥} | Slope | SE^{⊥} |
| 14:0/log phase | 0.58 | 0.03 | 0.54 | 0.01 | 0.56 | 0.02 |
| 14:0/stationary phase | 0.76 | 0.01 | 0.70 | 0.02 | 0.73 | 0.02 |
| 16:0/log phase | 0.61 | 0.03 | 0.55 | 0.02 | 0.58 | 0.03 |
| 16:0/stationary phase | 0.75 | 0.00 | 0.69 | 0.01 | 0.72 | 0.01 |

The “A and B” column shows the slopes and intercepts of the relationships when the data from the two sets of cultures were pooled. The R values were calculated from the δ values according to the equation δ = [(Rₛₐₘₚₗₑ/R_{Std}) − 1] * 1,000, where R_{std} = R_{VSMOW} = 0. 0.0001558.
^{⊥}SE: Standard error.

Metabolic Water: The isotopic distinction of intracellular water from extracellular water can be determined using a probe species. Approximately 53% of the hydrogen atoms from intracellular water in log-phase E. coli cells are isotopically distinct from extracellular water, these isotopically distinct hydrogen atoms being formed during metabolic processes. When the cells reached stationary phase, however, only 23% of the intracellular hydrogen atoms were derived from metabolism, indicating that the ability to maintain a large isotope gradient depends on the metabolic rate.

The methods described herein illustrate the calculation of the isotopic ratio of metabolically-derived hydrogen atoms in intracellular water. Interestingly, the hydrogen isotope ratio of metabolically formed water is −96% in log-phase cells, but δ²H is −179% in stationary-phase cells. It is important to note that metabolic water can consist of individual hydrogen and oxygen atoms within the pool of intracellular water molecules that did not originate as culture water, but rather were derived from metabolic reactions. The source of the hydrogen atoms in metabolic water detected in these experiments can be the hydrogen atoms in the nutrient molecules of the yeast extract and tryptone (an enzymatic hydrolysate of casein) supplied in the LB medium. Over the life cycle of a culture growing in LB, pools of specific substrate molecules can be depleted, so that the bacteria would be metabolizing different mixtures of molecules with potentially differing hydrogen isotope ratios at different times. Thus, one possible factor contributing to the difference in the δ²H values of metabolic water at log and stationary phases could be the changing substrate pools and accompanying changes in metabolic pathways.

Another factor that could contribute to the difference in apparent δ²H values of metabolic water at log and stationary phases is proton pumping, which can alter the hydrogen isotope ratio of intracellular water. The pKₐ for H₂O is 14.00, while that of D₂O is 14.9, indicating that D₂O tends to dissociate almost ten times less than H₂O. Thus, proton pumping might serve to enrich intracellular water by removing proportionally more protons than deuterons. If proton pumping is more active in log phase than in stationary phase, the isotope ratio of intracellular water in log-phase cells would be more enriched than that of stationary-phase cells, consistent with the data.

Isotopic Signature of Intracellular Water in Fatty Acids: The biosynthesis of saturated acyl fatty acids consists of repeated cycles of a 4-step process in which (1) an acetate unit is added to the growing acyl chain in a trans-acylation reaction, (2) the carboxyl group of the acetate moiety is reduced, (3) the resulting hydroxyl and a hydrogen from the adjacent carbon is removed to generate a double bond, and (4) the double bond is then reduced. Steps 2, 3, and 4 of this process comprise reactions in which hydrogen atoms are either added (steps 2 and 4) or removed (step 3) from the intermediate, and it is therefore expected that the hydrogen isotope ratio of fatty acids will be affected by the isotope ratio of the intracellular water at the time of biosynthesis. A complicating issue, however, is that hydrogen isotope fractionation at steps 2-4 will each contribute to the value of α_{water}, which represents the cumulative isotopic fractionation between culture water and the resulting fatty acid.

One interpretation of the regression coefficients in Table 4 is that they reflect a larger contribution of hydrogen atoms from culture water to the hydrogen atoms of fatty acids in stationary phase than in log phase. This interpretation assumes that the differences are not caused by differences in α_{water} between log and stationary phases. Isotopic fractionation in biochemical processes arises from unequal zero-point energies of bonds to heavy and to light isotopes resulting in different activation energies. Thus, fractionation factors (α) are a function of both temperature and the energetics of the individual enzyme-catalyzed reactions that comprise the pathway. The culture growth temperature was held constant throughout the experiments described herein, and consequently the value of α_{water} should not change if the enzymology of fatty acid biosynthesis remains constant. No evidence suggests that the enzymology of fatty acid biosynthesis differs between log and stationary phases, and it is therefore reasonable to presume that there is no difference in α_{water} between log and stationary phase.

The contribution of culture medium water to intracellular water increases as the culture progresses from log to stationary phase. Thus, hydrogen atoms in fatty acids from cells harvested during log phase will have a greater contribution from metabolic water than fatty acids harvested from cells in stationary phase. This prediction is consistent with the data illustrated in Table 4. Due to the number of unknown variables in Equation 4, the data in Table 4 cannot be used to directly calculate the fraction of intracellular water derived from metabolism. Nevertheless, these data confirm that the isotope ratios of metabolites can be used as indirect probes of metabolic rate in living cells.

Comparing Oxygen and Hydrogen Isotope Data: As noted above, approximately 53% of the hydrogen atoms found in intracellular water extracted from log-phase E. coli cells grown in 2×LB are isotopically distinct from extracellular water. Measuring the ¹⁸O/¹⁶O ratio of intracellular water from E. coli cells grown under the same conditions, however, yields that 71±19% of the oxygen atoms were isotopically distinct from growth medium water and were generated during metabolic processes. Both sets of data indicate that metabolically generated water is an important and substantial component of intracellular water in E. coli.

Two explanations can account for the difference percentage of hydrogen and oxygen atoms in intracellular water that is derived from metabolism. The first is that within experimental error (which were calculated from propagation of error in the slopes of the regressions, these numbers are not different at all. The second is that hydrogen atoms and oxygen atoms in metabolic water exchange with extracellular water at different rates. When water diffuses into or out of a cell either directly across the membrane or through aquaporin channels, both hydrogen and oxygen atoms are exchanged. These processes would therefore be expected to maintain parity between calculated percentages of intracellular water that is derived from metabolic processes using either hydrogen isotopes or oxygen isotopes. In addition to transport with water, however, hydrogen ions can also pass through membranes independently from oxygen atoms. The mechanisms by which protons can be transported across membranes include, but are not limited to, (1) proton permeation through membranes, (2) active transport via proton-purnping enzymes (e.g. cytochrome c oxidase), (3) diffusion via voltage-gated proton channels, and (4) diffusion via proton-permeable ion channels (e.g. gramicidin). Thus, a variety of pathways exist by which protons in metabolic water can exchange with extracellular water, many of which are not available to oxygen ions, and therefore there is no requirement that the percent of metabolic water calculated using these two different isotopes be equivalent. Nevertheless, the majority of intracellular water in log-phase E. coli cells is generated during metabolic processes is supported by both sets of data.

Sources of Hydrogen and Oxygen in Metabolic Water: An important facet in the generation of metabolic water is the initial source of the hydrogen and oxygen atoms. As discussed above, the source of the protons in metabolic water is the LB growth medium. The oxygen atoms, however, can have more than one potential source. In addition to the LB medium, oxygen atoms in metabolic water can also come from O₂ during respiration as the O₂ is reduced to water. A significant fraction of the water generated by the action of cytochrome c oxidase in Rhodobacter sphaeroides is released into the periplasmic space, which would rapidly equilibrate with extracellular water. However, even if cytochrome bo₃ of E. coli also releases water towards the “outside” of the cell, similar to R. sphaeroides, isotopically distinct intracellular water can exist in the periplasmic space. It is also possible that water released in this way diffuses or is transported back into the cytoplasm.

Another potential source of oxygen atoms in metabolic water is the LB medium. While both hydrogen and oxygen atoms in LB growth media can be released as water or otherwise solvent exchangeable atoms during biochemical processing, many of the “organic” oxygen atoms found in nutrients can be released as CO₂. The oxygen atoms in CO₂ can then exchange with intracellular water as a result of the enzymatic activity of carbonic anhydrase before the CO₂ diffuses to the atmosphere.

Potential Sources of Error: Both hydrogen and oxygen isotope ratios of water extracted from the cell cakes described in the Examples above are distinct from growth medium water. One mechanism that can produce isotopic changes in cell cake water is evaporation. However, evaporation usually results in an increase in heavy isotopes in the residual water, and the cell cakes described here were not uniformly enriched. While some samples were isotopically enriched relative to growth medium water, others sample were isotopically depleted. In addition, the slopes calculated from Equation 2 using both hydrogen and oxygen isotope data (see Table 3) are essentially identical. This provides additional evidence that evaporation is not a major artifact in our experiments because hydrogen and oxygen have different evaporative fractionations, and the two data sets would have had different slopes. While evaporative enrichment can be ruled out as a primary source of error, evaporation can potentially modulate the magnitude of the final results.

The proton pumping mentioned above would also cause the isotope ratio of intracellular water to be different from that of extracellular water. Again, however, the hypothesized isotopic enrichment due to proton pumping should cause the isotope ratio of intracellular water to be enriched compared to that of growth medium water in every sample. This was not observed. In addition, proton pumping cannot explain the data obtained for the oxygen isotope ratio of water extracted from cell cakes, which is consistent with hydrogen isotope ratio data.

Another mechanism that could cause the isotope ratios of extracted cell cake water to be different from the growth medium water is incomplete extraction of water from the cell cakes. Extraction of water from samples was accomplished by distillation in which the samples were heated under vacuum and the water vapor collected in a cold finger. Distillation follows Rayleigh kinetics, and if the process is incomplete, the remaining water is isotopically enriched while the distillate is isotopically depleted relative to the initial pool. The extracted cell cake water in the samples was not uniformly isotopically depleted relative to growth medium water. Furthermore, if a pool of non-exchangeable and unextractable water does exist in the cell, it would have to be isotopically distinct from growth medium water to account for our data.

The data herein is consistent with a two-component mixture, wherein the second component is metabolically-derived water. One possible source of a second, non-metabolic component is condensation. The cell cakes were stored at −20° C., and it is theoretically possible that sufficient condensation formed on the samples and/or tubes prior to extraction to alter the isotopic ratio of the cell cake water. However, the calculated isotope ratio of this second component (δ¹⁸O=−3.5%; δ²H=−96%) is not consistent with condensation of atmospheric water vapor. In addition, when the cells were chilled to 6° C. for 90 minutes prior to harvesting, the fraction of extracted cell cake water that was isotopically distinct from growth medium water was significantly reduced relative to cells that were not chilled. Because both the chilled and unchilled samples were the same size and were frozen and treated in an identical manner, it is expected that the amount of condensation that would form on the samples would be equal. The fact that the chilled cell cakes contained a significantly smaller fraction of isotopically distinct water, and the fact that the isotope ratios of this metabolically distinct water were inconsistent with meteoric water, provides evidence against the hypothesis that condensation forms the second water component.

The H:O ratios of the cell cake water as determined by TCEA indicate that if there are multiple components, that they must be cellular components that are volatile at physiological pH and either have the same H:O ratio as water or be present in relatively low abundance. It is important to note that both the hydrogen isotope ratio of extracted fatty acids described herein and the oxygen isotope ratio of isolated heme O suggest that intracellular water can be isotopically distinct from growth medium water, and the data herein suggests that the origin of this isotopically distinct water is metabolism. The apparently large fraction of metabolically-derived hydrogen atoms in intracellular water is surprising.

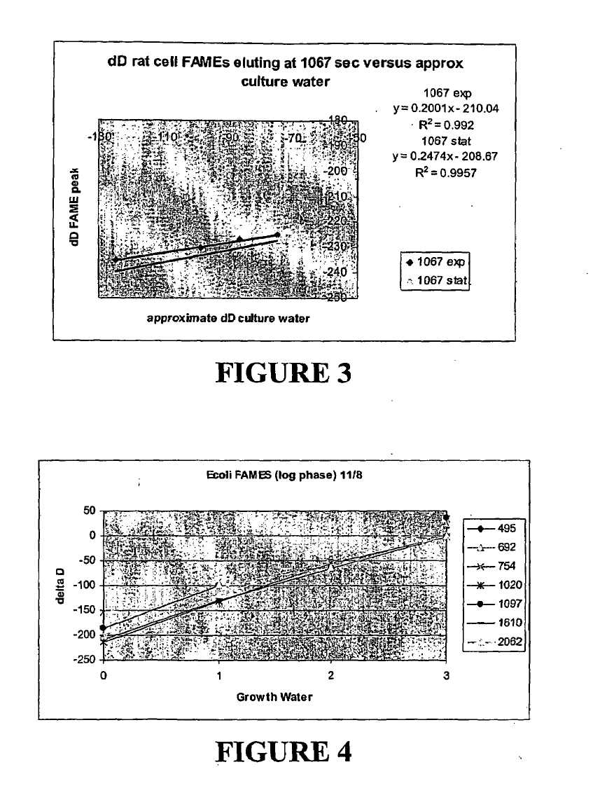

Similar tests and results have been performed with eukaryotic cells and are represented in FIG. 3.

### Example 3

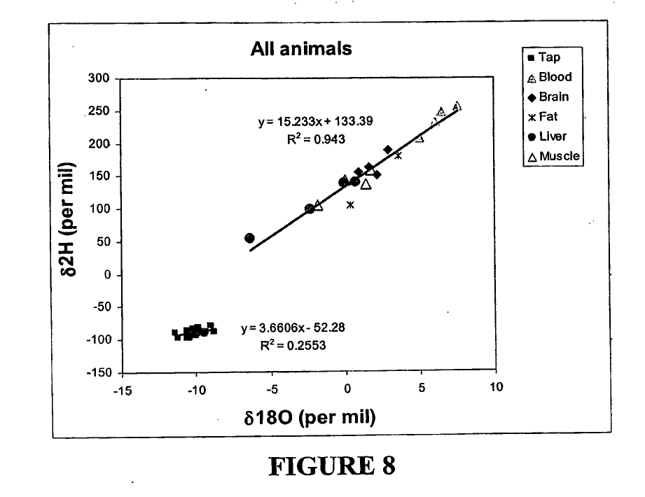

FIG. 8 illustrates data from experiments with eight different lab rats. Four of the lab rats were raised on Salt Lake City (SLC) tap water and the remaining lab rats were raised on slightly enriched water. Five different tissue samples were collected from each of the four rats. From the animals grown in slightly enriched water it can clearly be seen that the blood water has a very different isotope value for both O and H than the tissue water. The lab rats grown on SLC tap water follow the same trend but the values are much closer to each other (and are not separately labelled on this graph). This suggests that the isotope ratio of the metabolic water is reasonably close to that of SLC tap water. In the case of hydrogen, the signature of the food and tap water were similar thus masking the difference between metabolic water and extracellular water. Note, however, that tap water in other locations, such as, for example, Houston, can have different isotope ratios.

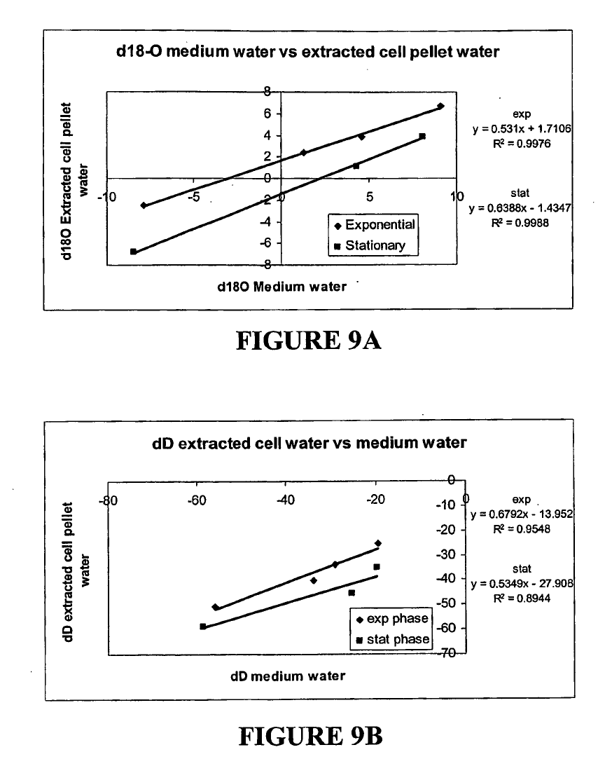

FIG. 9 illustrates rat fibroblasts harvested in either log or stationary phase. Cell cake water was extracted and both the H and O isotope ratio was determined. Significantly, the slope is much bigger in the stationary (“stat”) phase cells than in the log (“exp”) phase cells. This demonstrates that some of the water comes from metabolism, and that as metabolism slows down, the percentage of metabolic water in the intracellular water also decreases.

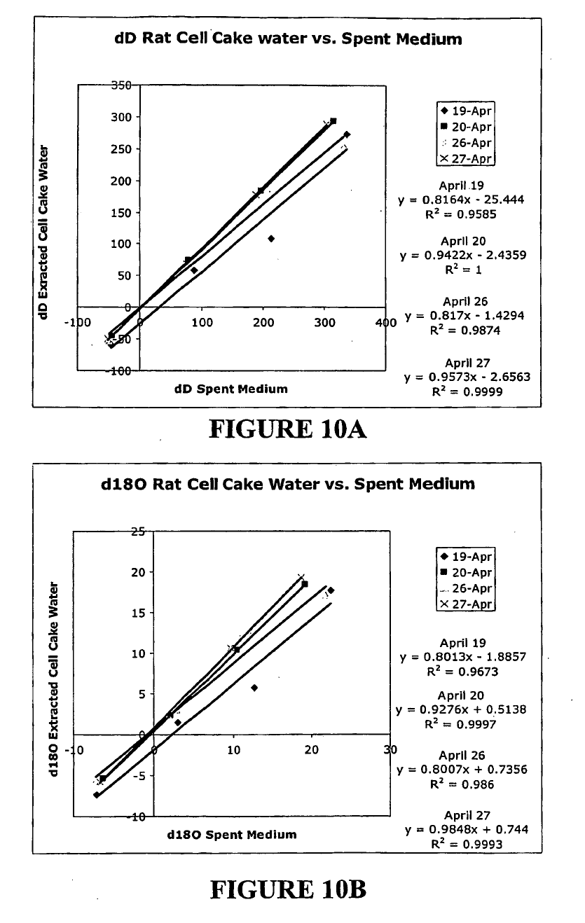

FIG. 10 illustrates a repeat of the experiment shown in FIG. 9 in duplicate. The diamonds and the triangles are both log phase cell data. The squares and cross are both stationary phase cell data. The log phase cells have a slope around 0.81 while the stationary phase cells have a slope around 0.95. Again, this indicates that stationary phase cells have less metabolic water in their intracellular water than do log phase cells.

Table 5 shows calculated amount of water in various tissue that comes from either food, O₂, or the water that the lab rats drink. Data from eight rats was used in this model calculation; four rats on tap water and four rats on slightly enriched water. Note that the sum of food plus O₂ gives the percent of the water from the tissue that is metabolic. Note also that this is a lower limit because this is total extracted water—it includes both intracellular water and extracellular water (i.e. blood). Finally, note that these values represent oxygen isotope values only.

TABLE 5
Amount of water in various tissue that comes from either
food, O₂, or the water that the lab rats drink

| TISSUE | Source | % |
|---|---|---|
| BRAIN | Food | 0.21 |
| | O₂ | 0.43 |
| | H₂O | 0.36 |
| | Metabolic | 0.64 |
| MUSCLE | Food | 0.22 |
| | O₂ | 0.45 |
| | H₂O | 0.33 |
| | Metabolic | 0.67 |
| LIVER | Food | 0.22 |
| | O₂ | 0.45 |
| | H₂O | 0.33 |
| | Metabolic | 0.67 |
| FAT | Food | 0.19 |
| | O₂ | 0.37 |
| | H₂O | 0.44 |
| | Metabolic | 0.56 |

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

### H. REFERENCES

Araguas-Araguas, L., Rozanski, K., Gonfiantini, R. & Louvat, D. (1995) J. Hydrol. 168, 159-171.

Booth, I. R., and Louis, P. (1999) Managing Hypoosmotic Stress: Aquaporins and Mechanosensitive Channels in Escherichia coli. Curr. Opin. Microbiol. 2, 166-169.

Brown, K. R., Brown, B. M., Hoagland, E., Manye, C. L., and Hegg, E. L. (2004) Heine A Synthase Does Not Incorporate Molecular Oxygen into the Formyl Group of Heme A. Biochemistry 43, 8616-8624.

Burgoyne, T. W., and Hayes, J. M. (1998) Quantitative Production of H-2 by Pyrolysis of Gas Chromatographic Effluents. Anal. Chem. 70, 5136-5141.

Calanita, G., Kempf, B., Bonhivers, M., Bishai, W. R., and Bremer, E. (1998) Regulation of the Escherichia coli Water Channel Gene aqpZ. Proc. Natl. Acad. Sci. U.S.A. 95, 3627-3631.

Chikaraishi, Y., Naraoka, H. & Poulson, S. R. (2004) Phytochemistry 65, 1369-1381.

Coplen, T. B. (1996) New Guidelines for Reporting Stable Hydrogen, Carbon and Oxygen Isotope-Ratio Data. Geochim. Cosmochim. Acta 60, 3359-3360.

Craig, H. (1961) Isotopic Variations in Meteoric Waters. Science 133, 1702-1703.

DeCoursey, T. E. (2003) Voltage-Gated Proton Channels and Other Proton Transfer Pathways. Physiol. Rev. 83, 475-579.

Devlin, T. M. (2005) Textbook of Biochemistry with Clinical Correlations, 6th ed., John Wiley & Sons, Hoboken, N.J.

Ehleringer, J. R., Casale, J. F., Lott, M. J. & Ford, V. L. (2000) Nature 408, 311-312.

Farquhar, G. D. & Cernusak, L. A. (2005) Funct. Plant Biol. 32, 293-303.

Farquhar, G. D., and Lloyd, J. (1993) Carbon and Oxygen Isotope Effects in the Exchange of Carbon Dioxide between Terrestrial Plants and the Atmosphere, in Stable Isotopes and Plant Carbon-Water Relations (Ehleringer, J. R., Hall, A. E., and Farquhar, G. D., Eds.) pp 47-70, Academic Press, San Diego.

Farquhar, G. D., Barbour, M. M. & Henry, B. K. (1998) in Stable Isotopes: Integration of Biological, Ecological, and Geochemical Processes, ed. Griffiths, H. (BIOS Scientific Publishers Ltd, Oxford), pp. 27-62.

Farquhar, G. D., Lloyd, J., Taylor, J. A., Flanagan, L. B., Syverstsen, J. P., Hubick, K. T., Wong, S. C. & Ehleringer, J. R. (1993) Nature 363, 439-443.

Garciá-Martin, M. L., Ballesteros, P., and Cerdan, S. (2001) The Metabolism of Water in Cells and Tissues as Detected by NMR Methods. Prog. Nucl. Magn. Reson. Spectrosc. 39, 41-77.

Gehre, M., Geilmann, H., Richter, J., Werner, R. A., and Brand, W. A. (2004) Continuous Flow 2H/1H and ¹⁸O/¹⁶O Analysis of Water Sarnples with Dual Inlet Precision. Rapid Commun. Mass Spectrom. 18, 2650-2660.

Haines, T. H. (1994) Water Transport Across Biological Membranes. FEBS Lett. 346, 115-122.

Handbook of Chemistry and Physics, 70th Edition; Weast, R. C., Lide, D. R., Astle, M. J., Beyer, W. H., Eds.; CRC Press: Boca Raton, Fla., 1990; p D-166.

Heath, R. J., and Rock, C. O. (1996) Roles of the FabA and FabZ Beta-Hydroxyacyl-Acyl Carrier Protein Dehydratases in Escherichia coli Fatty Acid Biosynthesis. I. Biol. Chem. 271, 27795-27801.

Hobson, K. A., Bowen, G. J., Wassenaar, L. I., Ferrand, Y. & Lormee, H. (2004) Oecologia 141, 477-488.

Ingraham, N. L. & Shadel, C. (1992) J. Hydrol. 140, 371-387.

Jencks, W. P. (1969) Catalysis in Chemistry and Enzymology, Dover Publications, New York.

Kendall, C., and Caldwell, E. (1998) Fundamentals of Isotope Geochemistry, in Isotope Tracers in Catchment Hydrology (Kendall, C., and McDonnell, J. J., Eds.) pp 51-86, Elsevier Science, New York.

Kreuzer-Martin, H. W., Ehleringer, J. R., and Hegg, E. L. (2005) Oxygen Isotopes Indicate Most Intracellular Water in Log-Phase Escherichia coli Is Derived from Metabolism. Proc. Natl. Acad. Sci. U.S.A. 102, 17337-17341.

Kreuzer-Martin, H. W., Lott, M. J., Dorigan, J. & Ehleringer, J. R. (2003) Proc. Natl. Acad. Sci. U.S.A. 100, 815-819.

Luo, Y.-H., Stemberg, L., Suda, S., Kumazawa, S., and Mitsui, A. (1991) Extremely Low D/H Ratios of Photoproduced Hydrogen by Cyanobacteria Plant Cell Physiol. 32, 897-900.

Mogi, T. (2003) in The Iron and Cobalt Pigments: Biosynthesis, Structure, and Degradation, eds. Kadish, K. M., Smith, K. M. & Guilard, R. (Academic Press, Amsterdam), Vol. 12, pp. 157-181.

Nagy, K. A. (1989) Doubly-Labeled Water Studies of Vertebrate Physiological Ecology, in Stable Isotopes in Ecological Research (Rundel, P. W., Ehleringer, J. R., and Nagy, K. A., Eds.) pp 270-286, Springer-Verlag, New York.

Potma, E. O., de Boeij, W. P., van Haastert, P. J. M., and Wiersma, D. A. (2001) Real-Time Visualization of Intracellular Hydrodynamics in Single Living Cells. Proc. Natl. Acad. Sci. U.S.A. 98, 1577-1582.

Rock, C. O., and Cronan, J. E. (1996) Escherichia coli as a Model for the Regulation of Dissociable (Type II) Fatty Acid Biosynthesis. Biochim. Biophys. Acta 1302, 1-16.

Rock, C. O., and Jackowski, S. (2002) Forty Years of Bacterial Fatty Acid Synthesis. Biochem. Biophys. Res. Commun. 292, 1155-1166.

Rock, C. O., St. Jude Children's Research Hospital. Private communication, 2006.

Sauer, P. E., Eglinton, T. I., Hayes, J. M., Schimmelmann, A. & Sessions, A. L. (2001) Geochim. Cosmochim. Acta 65, 213-222.

Schmidt, B., McCracken, J., and Ferguson-Miller, S. (2003) A Discrete Water Exit Pathway in the Membrane Protein Cytochrome c Oxidase. Proc. Natl. Acad. Sci. U.S.A. 100, 15539-15542.

Schweizer, E. (1989) Biosynthesis of Fatty Acids and Related Compounds, in Microbial Lipids (Ratledge, C., and Wilknson, S. G., Eds.) pp 3-50, Harcourt Brace Jovanovich, London.

Sessions, A. L., and Hayes, J. M. (2005) Calculation of Hydrogen Isotopic Fractionations in Biogeochemical Systems. Geochim. Cosmochim. Acta 69, 593-597.

Sessions, A. L., Burgoyne, T. W., and Hayes, J. M. (2001) Correction of H-3(+) Contributions in Hydrogen Isotope Ratio Monitoring Mass Spectrometry. Anal. Chem. 73,192-199.

Sessions, A. L., Burgoyne, T. W., and Hayes, J. M. (2001) Determination of the H-3 Factor in Hydrogen Isotope Ratio Monitoring Mass Spectrometry. Anal. Chem. 73, 200-207.

Sessions, A. L., Burgoyne, T. W., Schimmelmann, A. & Hayes, J. M. (1999) Org. Geochem. 30, 1193-1200.

Sessions, A. L., Burgoyne, T. W., Schimmelmann, A., and Hayes, J. M. (1999) Fractionation of Hydrogen Isotopes in Lipid Biosynthesis. Org. Geochem. 30, 1193-1200.

Sessions, A. L., Jahnke, L. L., Schimmelmann, A., and Hayes, J. M. (2002) Hydrogen Isotope Fractionation in Lipids of the Methane-Oxidizing Bacterium Methylococcus capsulatus. Geochim. Cosmochim. Acta 66, 3955-3969.

Shoemaker, D. P., Garland, C. W., and Nibler, J. W. (1989) Experiments in Physical Chemistry, 5th ed., McGraw-Hill, New York.

Silverman, D. N., and Lindskog, S. (1988) The Catalytic Mechanism of Carbonic Anhydrase: Implications of a Rate-Limiting Protolysis of Water. Acc. Chem. Res. 21, 30-36.

Sokal, R. R., and Rohlf, F. J. (1995) Biometry: The Principles and Practice of Statistics in Biological Research, 3rd ed., W.H. Freeman, New York.

Speakman, J. R., Visser, G. H., Ward, S. & Król, E. (2001) in Body Composition Analysis of Animals, ed. Speakman, J. R. (Cambridge University Press, Cambridge), pp. 56-98.

Speakman, J. R., Visser, G. H., Ward, S., and Król, E. (2001) The Isotope Dilution Method for the Evaluation of Body Composition, in Body Composition Analysis of Animals (Speakman, J. R., Ed.) pp 56-98, Cambridge University Press, Cambridge.

Stroud, R. M., Miercke, L. J. W., O'Connell, J., Khademi, S., Lee, J. K., Remis, J., Harries, W., Robles, Y. & Akhavan, D (2003) Curr. Opin. Struct. Biol. 13, 424-431.

Stroud, R. M., Nollert, P., and Miercke, L. (2003) The Glycerol Facilitator GlpF, Its Aquaporin Family of Channels, and Their Selectivity. Adv. Protein Chem. 63, 291-316.

Valentine, D. L., Chidthaisong, A., Rice, A., Reeburgh, W. S. & Tyler, S. C. (2004) Geochim. Cosmochim. Acta 68, 1571-1590.

Voet, D., and Voet, J. G. (2004) Biochemistry, 3rd ed., John Wiley & Sons, Hoboken, N.J.

Wiggins, P. M. (2001) High and Low Density Intracellular Water. Cell. Mol. Biol. 47, 735-744.

Wiggins, P. M. (2002) Water in Complex Environments Such as Living Systems.

Physica A 314, 485-491.

Yakir, D. (1992) Variations in the Natural Abundance of Oxygen-18 and Deuterium in Plant Carbohydrates. Plant, Cell Environ. 15, 1005-1020.

Yakir, D. (1992) Water Compartmentatin in Plant Tissue: Isotopic Evidence, in Water and Life: Comparative Analysis of Water Relationships at the Organismic, Cellular, and Molecular Levels (Somero, G. N., Osmond, C. B., and Bolis, C. L., Eds.) pp 205-222, Springer-Verlag, Berlin.

Zhu, X.-H., Lei, H., Zhang, Y., Zhang, X., Zhang, N., Ugurbil, K., and Chen, W. (2002) in Proc. Int. Soc. Mag. Res. Med. pp 1094, Hawaii.

Zhu, X.-H., Zhang, N., Zhang, Y., Zang, X., Ugurbil, K., and Chen, W. (2005) In vivo 17O NMR Approaches for Brain Study at High Field. NMR Biomed. 18, 83-103.

What is claimed is:

1. A method for determining the metabolic rate of a cell comprising
(a) obtaining a cell comprising a quantity of intracellular water, and
(b) analyzing at least a portion of the quantity of intracellular water to determine an isotopic composition of at least one of hydrogen or oxygen,
wherein the isotopic composition of at least one of hydrogen or oxygen is related to a known standard.

2. The method of claim 1, wherein the isotopic composition comprises a molar ratio of ²H/¹H.

3. The method of claim 1, wherein the isotopic composition comprises a molar ratio of ¹⁸O/¹⁶O.

4. The method of claim 1, wherein the isotopic composition comprises a molar ratio of ²H/¹H and a molar ratio of ¹⁸O/¹⁶O.

5. The method of claim 1, wherein the cell is bacterial.

6. The method of claim 1, wherein the cell is mammalian.

7. The method of claim 6, wherein the cell is obtained from a mammal selected from the group consisting of human, chimpanzee, monkey, cow, horse, pig, dog, cat, rat, guinea pig, and mouse.

8. The method of claim 1, wherein the cell is cancerous.

9. The method of claim 8, wherein the cancerous cell is selected from the group of cancers consisting of lymphomas (Hodgkins and non-Hodgkins), B cell lymphoma, T cell lymphoma, leukemias, myeloid leukemia, carcinomas, carcinomas of solid tissues, squamous cell carcinomas, squamotis cell carcinomas of the mouth, throat, larynx, and lung, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, mycosis fungoides, bladder cancer, brain cancer, nervous system cancer, lung cancers such as small cell lung cancer and non-small cell lung cancer, ovarian cancer, pancreatic cancer, prostate cancer, hepatic cancer, colon cancer, cervical cancer, cervical carcinoma, breast cancer, and epithelial cancer, renal cancer, genitourinary cancer, esophageal carcinoma, head and neck carcinoma, large bowel cancer, hematopoietic cancers, and testicular cancer.

10. A method for diagnosing an abnormal physical condition in an organism comprising:
(a) obtaining a probe comprising at least one hydrogen or oxygen resulting from a metabolic process of the organism,
(b) analyzing at least a portion of the probe to determine an isotopic composition of at least one of hydrogen or oxygen, and
(c) calculating the rate of the metabolic process using the isotopic composition of the at least one of hydrogen or oxygen,
wherein the rate of the metabolic process is related to a standard to provide a statistical probability for the existence of the abnormal physical condition.

11. The method of claim 10, wherein the probe is a fatty acid obtained from a mammalian subject, and wherein the analyzing comprises determining the isotopic composition of the hydrogen in at least a portion of the fatty acid.

12. The method of claim 11, wherein the mammalian subject is selected from the group consisting of human, chimpanzee, monkey, cow, horse, pig, dog, cat, rat, guinea pig, and mouse.

13. The method of claim 11, wherein the obtaining step further comprises isolating and methylating the fatty acid probe.

14. The method of claim 11, wherein the fatty acid is obtained from a mammalian blood sample.

15. The method of claim 10, wherein the isotopic composition comprises at least one rare isotope and at least one abundant isotope, and wherein the isotopic composition can be expressed as a molar ratio of the rare isotope to the abundant isotope.

16. The method of claim 10, wherein the probe is a prostate specific antigen.

17. The method of claim 10, wherein the abnormal physical condition is a cancer selected from the group of cancers consisting of lymphomas (Hodgkins and non-Hodgkins), B cell lymphoma, T cell lymphoma, leukemias, myeloid leukemia, carcinomas, carcinomas of solid tissues, squamous cell carcinomas, squamous cell carcinomas of the mouth, throat, larynx, and lung, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, mycosis fungoides, bladder cancer, brain cancer, nervous system cancer, lung cancers such as small cell lung cancer and non-small cell lung cancer, ovarian cancer, pancreatic cancer, prostate cancer, hepatic cancer, colon cancer, cervical cancer, cervical carcinoma, breast cancer, and epithelial cancer, renal cancer, genitourinary cancer, esophageal carcinoma, head and neck carcinoma, large bowel cancer, hematopoietic cancers, and testicular cancer.

18. The method of claim 10, wherein the abnormal physical condition is a weight disorder.
